(19) United States
(12) Patent Application Publication
(10) Pub. No.: US 2016/0279146 A1
(43) Pub. Date: Sep. 29, 2016

(54) METHODS AND PHARMACEUTICAL FORMULATIONS FOR TREATMENT OF SELECTIVE ESTROGEN-RECEPTOR MODULATOR-INDUCED ADVERSE DRUG REACTIONS

(71) Applicant: Limoxifen B.V., Almere (NL)

(72) Inventors: Jan Johan Adriaan TUITEN, Almere (NL); Konrad Bernardus Everardus BOCKER, Almere (NL); Hans Peter Frederik KOPPESCHAAR, Almere (NL)

(73) Assignee: Limoxifen B.V., Almere (NL)

(21) Appl. No.: 15/036,043

(22) PCT Filed: Nov. 11, 2014
(86) PCT No.: PCT/NL2014/050777

§ 371 Date: May 11, 2016

(30) Foreign Application Priority Data
Nov. 11, 2013 (EP) 13192325.2

(51) Int. Cl. A61K 31/568; A61K 45/06

(52) U.S. Cl. CPC A61K 31/568; A61K 45/06

(57) ABSTRACT

The invention related to methods of treatment of an individual suffering from or at risk of suffering from a selective estrogen-receptor modulator (SERM) induced adverse drug reaction. The invention additionally relates to a pharmaceutical dosage form, preferably a tablet, comprising a composition for pulsatile, not-continuous release of a compound or a combination of compounds that stimulate the dopamine and/or the noradrenergic system.

### FIELD

The invention relates to the field of medical treatment. It specifically relates to methods of treatment of an individual suffering from or at risk of suffering from a selective estrogen-receptor modulator (SERM)-induced adverse drug reaction. The invention further relates to pharmaceutical preparations that are suitable for use in these methods.

Estrogens play a key role in reproduction and have beneficial effects on the skeletal, cardiovascular, and central nervous systems in females. It is essential for normal sexual development and functioning of female organs that are important for childbearing, like the ovaries and uterus. It is necessary for the normal development of the breast. It also helps maintain the heart and healthy bones. However, it was found that women with a high lifetime exposure to estrogen are at higher risk for developing breast cancer.

Most estrogenic responses are mediated by estrogen receptors (ERs), either ERα or ERβ. About 75% of all breast cancers are estrogen receptor positive. They grow in response to the hormone estrogen, and are often dependent on estrogen for proliferation. In the absence of estrogen, the estrogen receptor (ER) is normally localized in the cytosol. Upon activation by estrogen, the ER/hormone complex migrates to the nucleus, dimerizes and binds to specific sequences of DNA known as hormone response elements. Following recruitment of other proteins to the DNA-bound receptor/hormone complex, the expression of genes that are functionally coupled to the hormone response elements is modulated. The composition of the final complex determines the activity of the estrogen receptor.

The expression of ERα and ERβ, and the expression of coactivators and corepressors, differ between cell types. Due to these differences, estrogen-like compounds may interact differently with a receptor/hormone complex in different cell types. Such estrogen-like compounds are called selective estrogen receptor modulators, or SERMs. Dependent on the target tissue, a SERM selectively agonizes or antagonizes the activity of an estrogen receptor complex.

For example, a SERM can inhibit an estrogen receptor found in breast cells but activate an estrogen receptor present in uterine endometrial cells. A SERM of this type would inhibit cell proliferation in breast cells, but stimulate the proliferation of uterine endometrial cells. The SERMs tamoxifen, endoxifen and raloxifene exhibit ER antagonist activity in breast and agonist activity in bone. However, only tamoxifen exhibits agonist activity in the uterus.

SERMs such as tamoxifen are used for treatment of ER-positive breast cancer patients. In general, SERM-comprising endocrine therapy is given when surgery, and/or radiation therapy are finished. SERM-comprising endocrine therapy is generally accepted to prevent recurrence of the cancer by blocking the proliferation stimulating effects of estrogen on the cancer cells.

Tamoxifen may be taken by women for up to five years after initial treatment for breast cancer. Since its approval in 1998, tamoxifen has been used to treat millions of women and also men that were diagnosed with ER-positive breast cancer. Adverse drug effects include endometrial abnormalities such as hyperplasia, metaplasia, atypical hyperplasia, and endometrial polyps. Further adverse effects that have been reported include depression, nausea, vision problems and vaginal bleeding (Demissie et al., 2001. J Clin Oncol 19: 322-328). Patients who experienced adverse drug effects are more likely to stop taking a SERM such as tamoxifen (Wouters et al., 2013. Annals of Oncology 24:2324-2329).

A few studies have focused on the epidemiology and treatment of SERM-induced distress and mixed depressive states (Fan et al., 2008. Gen Hosp Psych 30: 112-126). Selective serotonin reuptake inhibitors (SSRIs) and serotonin-norepinephrine reuptake inhibitors are often prescribed for treatment of depression, including depression occurring in tamoxifen-treated patients. It was found, however, that most SSRIs inhibit CYP2D6, a member of the cytochrome P450 mixed-function oxidase system, which is required for the generation of endoxifen, the active metabolite of tamoxifen (Jin et al., 2005. J Natl Cancer Inst 97: 30-39). Due to this drug-drug interaction, considerably reduced endoxifen concentrations by inhibition of CYP2D6 are the result. Hence, it was strongly recommended to avoid CYP2D6-inhibiting antidepressants (e.g., paroxetine, fluoxetine) in patients treated with tamoxifen for breast cancer (Binkhorst et al., 2013. Breast Cancer Res Treat. 139: 923-929).

The present invention describes new methods of treatment of ER-positive cancer patients that overcomes at least in part the adverse drug effects that are associated with SERM-containing regimens. Said methods result in better compliance of patients with SERM-containing regimens such as tamoxifen and, thus, more favorable outcomes.

The present invention therefore provides a method of treatment of an individual suffering from, or at risk of suffering from, a selective estrogen receptor modulator (SERM)-induced adverse drug reaction, said method comprising administering to the individual in need thereof a compound that stimulates the dopamine and/or the noradrenergic system in the individual. Said compound preferably does not inhibit CYP2D6. It is further preferred that the compound is administered to the individual before or, more preferred, during the period that the patient is treated with the SERM.

The term “selective estrogen receptor modulator (SERM)”, as is used herein, refers to a class of compounds that act on the estrogen receptor. The action of a SERM differs in various tissues, thereby selectively inhibiting or stimulating estrogen-like action in tissues.

A SERM-induced adverse drug reaction includes depression, decreased sexual desire, cognitive side effects such as decreased verbal memory, decreased processing speed, decreased executive functioning; and/or arthralgic symptoms such as joint and muscle pain. A SERM-induced adverse drug reaction that is preferably treated with a compound that stimulates the dopamine and/or the noradrenergic system is depression.

Depression is a serious mental health problem that has increased in prevalence over the years. Depression is estimated to affect over 17 million inhabitants on the United States each year. The socioeconomic impact of depression is significant; the cost is estimated to be over $44 billion annually. Further, people suffering from depression carry the risk of suicide. Up to 15% of those who are suffering from depression the by suicide. There are 30,000 to 35,000 suicide-related deaths attributed to depression a year, which is a rate similar to the death rate from leukemia.

The term “depression”, as is used herein, refers to clinical depression, also known as major depression or major depressive disorder

A preferred compound that stimulates the dopamine and/or the noradrenergic system in an individual is a catechol-O-methyltransferase (COMT) inhibitor.

The term “COMT inhibitor”, as is used herein, refers to a drug that inhibits the action of catechol-O-methyl transferase. This enzyme catalyzes the meta-O-methylation of adrenaline, noradrenaline and their deaminated metabolites in the liver and the kidney, and metabolizes locally released noradrenaline in effector tissue. COMT inhibitors are used in the treatment of Parkinson's disease.

Preferred COMT inhibitors include nitecapone (3-(3,4-dihydroxy-5-nitrobenzylidene)-2,4-pentanedione), which can be orally administered at 2-200 mg/kg, tolcapone (3,4-dihydroxy-4′-methyl-5nitrobenzophenone), which can be orally administered at 10-2000 mg/day, and entacapone ((E)-2-cyano3-(3,4-dihydroxy-5-nitrophenyl)-N,N-diethyl-2-propenamide), which can be orally administered at 10-1600 mg/day.

A further preferred compound that stimulates the dopamine and/or the noradrenergic system in an individual is selected from an estrogen, preferably estradiol; 3α-androstanediol (5α-androstane-3α,17β-diol); a nootropic such as a dopamine agonist, including amisulpride ((RS)-4-amino-N-[(1-ethylpyrrolidin-2-yl)methyl]-5-ethylsulfonyl-2-methoxy-benzamide), which blocks dopamine autoreceptors at low dosages thereby resulting in higher concentrations of dopamine in the central nervous system, ropinirole (4-[2-(dipropylamino)ethyl]-1,3-dihydro-2H-indol-2-one), which is an agonist of D2, D3, and D4 dopamine receptors, pramipexole ((S)-2-amino-6-(propylamino)-4,5,6,7-tetrahydrobenzothiazool) which is an agonist of D2, D3 and D4 dopamine receptors, L-phenylalanine, L-tyrosine, N-acetyl-L-tyrosine, and/or L-3,4-dihydroxyphenylalanine, which are metabolic precursors of catecholamines such as dopamine; biopterin and/or pyridoxal-phosphate (PLP, pyridoxal-5′-phosphate or P5P), which are involved in the biosynthesis of dopamine, and further compounds that are used for the treatment of Parkinson disease such as bromocriptine (5′α)-2-bromo-12′-hydroxy-5′-(2-methylpropyl)-3′,6′,18-trioxo-2′-(propan-2-yl)ergotaman), cabergoline (N-[3-(Dimethylamino)propyl]-N-[(ethylamino)carbonyl]-6-(2-propenyl)-8g-ergoline-8-carboxamide), dihydrexidine (6,6a,7,8,12b-hexahydro-benzo(a)phenanthridine-10,11-diol), dinapsoline ((8,9-dihydroxy-2,3,7,11b-tetrahydro-1H-naph[1,2,3-de]isoquinoline), doxanthrine ((6aS,12bR)-6a,7,8,12b-tetrahydro-6H-chromeno[3,4-c]isoquinoline-2,3-diol), e epicriptine (2R,4R,7R)—N-[(1S,2S,4R,7S)-2-hydroxy-7-(S)-(1-methylpropyl)-5,8-dioxo-4-(propan-2-yl)-3-oxa-6,9-diazatricyclo [7.3.0.02,6]dodecan-4-yl]-6-methyl-6,11-diazatetracyclo [7.6.1.02,7.012,16]hexadeca-1(16),9,12,14-tetraene-4-carboxamide), lisuride 1,1-Diethyl-3-(7-methyl-4,6,6a,7,8,9-hexahydro-indolo[4,3-fg]quinolin-9-yl)-urea), pergolide ((8(3)-8-[(methylthio)methyl]-6-propylergoline), piribedil (2-[4-(benzo[1,3]dioxol-5-ylmethyl)piperazin-1-yl]pyrimidine), pramipexole ((S)-2-amino-6-(propylamino)-4,5,6,7-tetrahydrobenzothiazool), rotigotine ((S)-6-[propyl(2-thiophen-2-ylethyl)amino]-5,6,7,8-tetrahydronaphthalen-1-ol), roxindole (3-[4-(4-phenyl-3,6-dihydro-2H-1-pyridin-1-yl)butyl]-1H-indol-5-01), and sumanirole ((R)-5,6-dihydro-5-(methylamino)-4H-imidazo[4,5,1-ij]quinolin-2(1H)-one).

Yet a further preferred compound that stimulates the dopamine and/or the noradrenergic system in an individual is a male steroid, preferably a high androgenic steroid.

The term “male steroid”, as is used herein, refers to a natural or synthetic steroid hormone that is able to bind the androgen receptor. The term includes androgenic steroids such as testosterone and dihydrotestosterone, androstenedione, and anabolic steroids such as testolactone, methyltesterone, fluoxymesterone, clostebol, formestan, methandriol dipropionate, methandrostenolone, methenolone, oxabolone and oxymesterone. Anabolic steroids are described in “Sean C. Sweetman, ed (2009). Martindale: The Complete Drug Reference (36th edition ed.). Pharmaceutical Press, London, GB and/or Llewellynn W, (2009). Anabolics 9^{th} Edition. Molecular Nutrition, LLC. Jupiter (Fla.), USA. The indicated anabolic steroids can be obtained commercially, for example as Dianabol (methandrostenolone), Primobolan (methenolone) and Oranabol (oxymesterone).

The term “high androgenic steroid”, as used herein, refers to a steroid of which androgenic effects are at least similar or higher than anabolic effects. Androgenic effects include induction of male phenotype, growth of sexual organs, development of secondary sexual characteristics, maintenance of sexual function, and fertility. Anabolic effects include nitrogen retention and increases in muscle mass and strength. A test to compare androgenic and anabolic effects of an androgenic steroid is provided by the Hershberger Bioassay (Hershberger et al., 1953. Proc Soc Exp Biol Med 83:175-80). According to this bioassay, an increase in weight of prostate/seminal vesicles is compared with an increase in weight of the levator ani muscle in castrated rats as indicator of androgenicity and anabolic activity, respectively. Testosterone is one of the most potent anabolic steroids and it may be impossible to separate the two activities (anabolic, nitrogen-sparing effects and androgenic, virilizing effects) completely. Although androgens mediate a broad range of developmental and homeostatic function (including psychoactive effects), all androgens induce their response via a single androgen receptor, despite this diversity.

A preferred high androgenic steroid is testosterone and/or dihydrotestosterone.

Yet a further preferred male steroid is a high anabolic steroid. Said high anabolic steroid is preferably selected from the group consisting of testolactone, methyltesterone, fluoxymesterone, clostebol, formestan, methandriol dipropionate, methandrostenolone, methenolone, oxabolone and oxymesterone. Unlike testosterone, these anabolic steroids are not converted by aromatase into estrogens such as estradiol (Meinhardt and Mullis, 2002. Seminars in Reproductive Medicine 20: 277-284). Thus, use of a high anabolic steroid according to the invention provides an advantage in that undesirable increases of estrogen levels are prevented.

The anabolic steroids in the pharmacological compositions according to the invention are preferably not administered by injection. Unlike most anabolic steroids, testolactone, methyltesterone, fluoxymesterone, clostebol, formestan, methandriol dipropionate, methandrostenolone, methenolone, oxabolone and oxymesterone can be taken orally and do not require invasive routes of administration, such as intramuscular or intraperitoneal injection. Oral administration avoids influencing the motivation for sexual behaviour in a negative manner, what would most likely occur in case of an invasive route of administration such as an injection.

A male steroid is preferably administered in the form of a patch or gel, for example for continuous delivery. Said patch may contain a permeation enhancer. A preferred daily patch comprises 0.25-10 mg of a high androgenic or high anabolic steroid. The patch is preferably applied at nighttime to the abdomen, upper arms, back, or upper thighs. Said steroid gel preferably comprises about 1% male steroid and is preferably applied to the shoulder, abdomen, or upper arm after bathing. About 10% of the steroid is absorbed into the stratum corneum of the skin, which serves as a reservoir for the male steroid, allowing its slow release over several hours. When applied as a gel, male steroid levels reach a steady state in 1 to 2 days.

A male steroid can also be delivered orally as a steroid ester able to pass the liver and result in systemic blood levels of the steroid.

A steroid inhaler preferably comprises between 0.1-0.5 mg of a male steroid. Administration by inhalation results in a “pulse” profile with a rapid return to baseline levels.

Testosterone and/or dihydrotestosterone are preferably administered in the form of a sublingual formulation, such as a sublingual formulation comprising cyclodextrins as an inclusion complex forming carrier. Another example of a suitable route of administration is buco-mucosally or intranasally, or through an inhaler, which can also be performed with the use of a cyclodextrin-containing formulation or other common excipients, diluents and the like that are known in the art. A typical example of a formulation is given in hydroxypropyl-beta cyclodextrin, but other cyclodextrins and other common excipients, diluents and the like are within the skill of the art for preparing a formulation comprising testosterone or an analogue thereof. It is preferred that the pharmaceutical product is designed for sublingual administration and even more preferred said composition comprises a cyclodextrin such as hydroxypropyl-beta cyclodextrin. A typical example of a liquid testosterone sample (for 0.5 mg of testosterone) consists of 0.5 mg testosterone, 5 mg hydroxypropyl-betacyclodextrines (carrier), 5 mg ethanol, and 5 ml water, but each of the amounts of these substances might be higher or lower.

A male steroid is preferably administered such that the circadian rhythm of testosterone is mimicked. The regulation of androgen synthesis is complex and more critical in females than in males. Androgen excess in females results in a variety of pathological conditions, ranging in severity from acne/virilization during puberty or disturbance of menstrual cycles in adult woman to polycystic ovary syndrome (PCOS) with infertility, hirsutism, obesity, and insulin resistance (Ankarberg and Norjavaara, 1999. J Clin End & Metab 84: 975-984). Testosterone levels in males and females are highest in the morning, usually between 7-8 and 11 a.m. and gradually decline thereafter resulting in lowest levels of testosterone in the evening. It is thought that this circadian rhythm is critical for the well-being of an individual.

The administration of a compound that stimulates the dopamine and/or the noradrenergic system in the individual, preferably of a male steroid, preferably comprises the daily administration at a specific time of the day, preferably in the morning, of a composition that is designed for immediate (peak) release of a first active agent followed by rapid absorption into the bloodstream, and the administration of a composition for time-delayed immediate or sustained release of a second active agent, wherein the first and second active agent comprises said male steroid, preferably testosterone and/or dihydrotestosterone.

The amount of a male steroid per pharmaceutical composition that is provided as an immediate peak release formulation is preferably 0.05-20 mg; preferably 0.1-10 mg; more preferably 0.2-5 mg. In a particularly preferred embodiment the amount of male steroid released per pharmaceutical composition is 0.3-2.5 mg, preferably about 0.5 mg male steroid. The amount of male steroid that is provided as an immediate peak release formulation preferably results in a peak plasma level of free male steroid of about at least 0.010 nmol/L, which will typically occur between 1 and 20 minutes after administration of the male steroid.

The amount of male steroid that is provided as a time-delayed immediate or sustained release formulation is preferably 0.05-20 mg; preferably 0.1-10 mg; more preferably 0.2-5 mg. In a particularly preferred embodiment the amount of male steroid in the time-delayed immediate or sustained release formulation is 0.3-2.5 mg, preferably about 0.5 mg male steroid.

A sustained release formulation or composition of the invention is preferably but not necessarily a time-delayed, sustained release formulation, preferably in combination with a pH-insensitive coating. The time delay formulation allows release of the active ingredients after a defined time delay. A suitable time-delay formulation according to the present invention preferably additionally comprises an enteric coating.

Said individual suffering from or at risk of suffering from a selective estrogen-receptor modulator (SERM) induced adverse drug reaction typically is an individual in the course of a breast cancer treatment.

Breast cancer most commonly originates from the inner lining of milk ducts or the lobules that supply the ducts with milk. Cancers originating from ducts are known as ductal carcinomas, while those originating from lobules are known as lobular carcinomas. Breast cancer occurs in humans and other mammals. While the overwhelming majority of human cases occur in women, male breast cancer also occurs.

There are presently two main chemical classes of SERMs that are used in the clinic. These include the triphenylethylene derivatives tamoxifen and toremifene for treatment of breast cancer, and raloxifene, a benzothiopene derivative for treatment and prevention of osteoporosis and in the USA for the prevention of breast cancer. Said breast cancer is preferably typed as an estrogen receptor positive cancer. SERM-containing treatment alone is thought to be adequate for premenopausal women with ER-positive, low-risk cancers. Low risk cancers are defined as node-negative, ER/PR-positive, small (less than 1 cm) and lacking unfavorable microscopic features (Goldhirsch et al., 2007. Ann Oncol 18: 1133).

Said individual preferably is a female breast cancer patient, preferably a premenopausal breast cancer patient. SERMs such as tamoxifen have anti-estrogenic effects on the premenopausal vagina and may cause vaginal atrophy, dryness, and dyspareunia, which will influence the individual's mood and interest in activities.

Said individual may be suffering from or at risk of suffering from breast cancer metastasis. Individuals with ER-positive metastases are preferably treated with second-line hormone therapy, which includes a selective aromatase inhibitor such as anastrozole (2,2′-[5-(1H-1,2,4-triazol-1-ylmethyl)-1,3-phenylene]bis(2-methylpropanenitrile), letrozole (4,4′-((1H-1,2,4-triazol-1-yl)methylene)dibenzonitrile), and/or exemestane (6-methylideneandrosta-1,4-diene-3,17-dione), and/or the SERM fulvestrant. The SERM-induced adverse drug reaction is thought to be increased when a SERM such as tamoxifen is combined with an aromatase inhibitor and/or with the SERM fulvestrant.

Uptake from plasma of estrogens that is synthesized in the ovary is the primary mechanism for maintenance of estradiol concentrations in breast cancer tissue in premenopausal women. However, several steps may be operant in postmenopausal women. These include enzymatic synthesis of estradiol via sulfatase, aromatase, and 17β-hydroxysteroid dehydrogenase in the tumor itself. Aromatization of androgens secreted by the adrenal to estrogens in peripheral tissues and transport to the tumor via circulation in the plasma provides another means of maintaining breast tumor estradiol levels in postmenopausal women. These various sources contribute to the high tissue estrogen levels measured in breast tumor tissue. Aromatization is inhibited by administration of an aromatase inhibitor, either alone or combined with a SERM such as tamoxifen.

Fulvestrant is an estrogen receptor antagonist with no agonist effects, which works by down-regulating the estrogen receptor. It is administered as a once-monthly injection.

A method according to the invention preferably further comprises the diagnoses of depression in said individual prior to and/or during said treatment. Currently, depression is diagnosed by verbal tests that must be administered by a mental health professional. The tests rely on self-reports from the subject whereby the subjects verbally describe their feelings or are presented with verbally-described scenarios and select the scenario that best describes their feelings. The clinician then uses these responses to rate the symptoms. The rating scales provide check-lists for clinicians and diagnosticians to monitor patients' responses to treatment or reactions to environmental changes (see also U.S. Pat. No. 8,394,354, which is included herein by reference).

Said diagnosis of depression preferably comprises decreased mood and/or decreased sexual desire and/or cognitive side effects such as decreased verbal memory, decreased processing speed, decreased executive functioning and/or muscle ache. According to the DSM-IV and the DSM-V criteria for diagnosis of major depressive disorder, at least 5 symptoms selected from depressed mood, marked diminished interest or pleasure, significant weight loss or weight gain, insomnia or hypersomnia, psychomotor agitation or retardation, fatigue or loss of energy, feelings of worthlessness or excessive guilt, diminished ability to concentrate, and recurrent thoughts of death or suicidal ideation, have been present during a 2-week period and include either depressed mood or loss of interest or pleasure.

Known diagnostic tests that can be used for diagnosing depression include Goldberg's depression test, Hamilton Depression Rating Scale (HDRS), Beck Depression Inventory (BDI), Patient Health Questionnaire (PHQ), Major Depression Inventory (MDI), Center for Epidemiologic Studies Depression Scale (CES-D), Zung Self-Rating Depression Scale (SDS), Geriatric Depression Scale (GDS) and Cornell Scale for Depression in Dementia (CSDD), which are all known to the skilled person. In addition, suitable diagnostic tests have been described in the literature, for example by Williams et al., 2002. Gen Hosp Psychiatry 24: 225-237, which is included herein by reference.

A preferred SERM that induces an adverse drug reaction includes a SERM that is an estrogen antagonist in breast tissue, especially breast cancer tissue. A preferred SERM is fulvestrant (7α,17β)-7-{9-[(4,4,5,5,5-pentafluoropentyl)sulfinyl]nonyl}estra-1,3,5(10)-triene-3,17-diol), which is an estrogen receptor antagonist with no agonist effects and can be administered as a once-monthly injection at 200-500 mg; raloxifene ([6-hydroxy-2-(4-hydroxyphenyl)-benzothiophen-3-yl]-[4-[2-(1-piperidyl)ethoxy]phenyl]-methanone), which can be orally administered at 60-240 mg/kg/day; lasofoxifene ((5R,6S)-6-phenyl-5-[4-(2-pyrrolidin-1-ylethoxy)phenyl]-5,6,7,8-tetrahydronaphthalen-2-ol), which can be orally administered at 0.001 mg/kg-1.0 mg/kg/day, and triphenylethylene derivatives such as tamoxifen ((Z)2-[4-(1,2-diphenyl-1-butenyl) phenoxy]-N, N-dimethylethanamine 2-hydroxy-1,2,3-propanetricarboxylate (1:1)), which can be orally administered at 20 to 200 mg/kg per day, and/or toremifene (2-{4-[(1Z)-4-chloro-1,2-diphenyl-but-1-en-1-yl]phenoxy}-N,N-dimethylethanamine), which can be orally administered at 10 to 800 mg/day.

A most preferred SERM is or comprises tamoxifen and/or it's active metabolite endoxifen.

The term “tamoxifen” includes the compound tamoxifen ((Z)-2-[4-(1,2-diphenylbut-1-enyl)phenoxy]-N,N-dimethyl-ethanamine), and metabolites thereof such as 4-hydroxytamoxifen ((Z)-4-(1-(4-(2-(dimethylamino)ethoxy)phenyl)-2-phenylbut-1-enyl)phenol) and 4′-hydroxytamoxifen ((Z)-4-(1-[4(dimethylaminoethoxy)phenyl]2-phenyl-1-butenyl)phenol).

Especially when a male steroid, preferably a high androgenic steroid such as testosterone, is used as a compound that stimulates the dopamine and/or the noradrenergic system in the individual in a method according the invention, it is preferred that the individual is further receiving an aromatase inhibitor. As an alternative, a non-aromatisable steroid such as dihydrotestosterone, may be used. Said aromatase inhibitor preferably is a selective aromatase inhibitor such as anastrozole, which is preferably orally administered at between 0.2 and 5 mg/day, preferably about 1 mg/day, letrozole, which is preferably orally administered at between 0.2 and 10 mg/day, preferably about 2.5 mg/day and/or exemestane, which is preferably orally administered at between 2 and 200 mg/day, preferably about 25 mg/day. Said aromatase inhibitor inhibits and/or prevents aromatization of androgenic steroids such as testosterone into estradiol, resulting in higher effective levels of the high androgenic steroid such as testosterone for stimulating the dopamine and/or the noradrenergic system in the individual.

The invention further provides a compound or a combination of compounds that stimulate the dopamine and/or the noradrenergic system for use in a method for the treatment of an individual suffering from, or at risk of suffering from, a selective estrogen-receptor modulator (SERM)-induced adverse drug reaction, including depression, decreased sexual desire, cognitive side effects such as decreased verbal memory, decreased processing speed, decreased executive functioning; and/or arthralgic symptoms such as joint and muscle pain, preferably depression. Said compound or combination of compounds preferably is or comprises a high androgenic steroid, preferably testosterone and/or dihydrotestosterone.

The invention further provides the use of a compound or a combination of compounds that stimulate the dopamine and/or the noradrenergic system for the manufacture of a medicament for use in treatment of an individual suffering from, or at risk of suffering from, a selective estrogen-receptor modulator (SERM)-induced adverse drug reaction, including depression, decreased sexual desire, cognitive side effects such as decreased verbal memory, decreased processing speed, decreased executive functioning; and/or arthralgic symptoms such as joint and muscle pain, preferably depression. Said compound or combination of compounds preferably is or comprises a male steroid, preferably a high androgenic male steroid, preferably testosterone and/or dihydrotestosterone.

The invention further provides a pharmaceutical dosage form comprising a composition for pulsatile, not-continuous release of a compound or a combination of compounds that stimulate the dopamine and/or the noradrenergic system. Pulsatile drug delivery systems that are administered via the oral route include time controlled drug delivery systems. Such dual drug delivery devices are designed to release a drug at two different periods of time. For example, a drug is quickly released in a first phase to provide maximum relief within a short time frame, which is followed by a sustained release phase to avoid a need for repeated frequent administration and/or to mimic the circadian rhythm of steroids.

The skilled person will understand that a drug with low oral bioavailability due to, for example, a low diffusion rate, a low permeation rate and/or a high efflux rate, will by itself result in a sustained release phase without specific formulation after oral administration. The term diffusion, as is used herein, refers to the diffusion of a drug through an aqueous environment. The term permeation, as used herein, refers to the permeation through the intestinal wall by passive diffusion and/or active transport. The term efflux, as used herein, refers to the efflux of a drug back into the intestinal lumen which reduces the net absorption of the drug.

Suitable devices for use as a biphasic release system are compressed double-layer tablets and “core-within-coating” systems, which involves the use of a sustained release tablet as a compressed core that is coated over the whole surface with a disintegrating formulation. Both the core tablet and the outer coating contain a drug. A simple pulsatile formulation is a two layer press coated tablet consisting of polymers with different dissolution rates.

Some biphasic release devices exist in the art. WO93/009771 describes a two pulse tablet of flutamide for the treatment of prostate cancer. The first pulse is obtained from an immediate release layer while the second pulse is obtained from a core which contains a solid dispersion of the flutamide in a carrier. The immediate release layer and the core are separated by a film layer of an enteric coating.

Multiparticulates also provide a biphasic release system. WO94/12160 describes a capsule which contains a plurality of pellets with varying delay times to drug release. By mixing pellets of different delay times one can obtain pulsatile delivery of the drug. The drug is contained in the pellet along with an osmotic ingredient. The pellets are coated with a water permeable, water-insoluble film that allows water diffusion into the pellet. The osmotic ingredient dissolves in the water causing the pellet to swell and eventually burst to release drug. The osmotic ingredient that is contained in a pellet, and the coating of pellet, are two of the variables that determine the delay time of a drug that is contained in a pellet.

In addition, a pulsatile system that is based on multiple particles is described in WO 98/51287. The drug release from the particles is controlled by combinations of controlled release layers, swelling layers and coating layers. The controlled release layer is a crosslinked poly (acrylic acid) polymer of high molecular weight admixed with a water soluble polymer.

A preferred pulsatile drug delivery system is a chrono-pharmaceutical drug delivery system that matches human circadian rhythms. Such pulsatile drug delivery systems are known in the art. For example, several of such systems are described in Kalantzi et al., 2009. Recent Patents on Drug Delivery & Formulation 3: 49-63. Further examples are provided in WO2012/158030. Both Kalantzi et al., 2009 and WO2012/158030 are included herein by reference.

A preferred pharmaceutical dosage form comprises a composition for the immediate peak release of a first active agent and a composition for release of second active agent. Said composition for release of second active agent preferably comprises multiple subunit compositions, for example different layers, that provide repeated release of the second active agent, such as a second release, a second and a third release, or a second, a third release, and a fourth release of the second active agent.

A further preferred pharmaceutical dosage form comprises a composition for the immediate peak release of a first active agent and a composition for time-delayed, immediate or sustained, release of a second active agent.

Yet a further preferred pharmaceutical dosage form comprises a composition for the immediate oral, pulmonary or nasal release of a first active agent and a composition for time-delayed, immediate or sustained, release of a second active agent.

The invention further provides a tablet comprising a composition for immediate sublingual release of a first active agent and a composition for time-delayed, immediate or sustained, release of a second active agent. Said composition for time-delayed immediate or sustained release is preferably based upon pharmaceutical delivery at different pH values, explosion-controlled drug delivery systems, time delayed diffusion controlled rupturing dosage forms, and/or colonic drug delivery systems. Preferred examples of such delivery systems have been described in WO2012/158030, which is hereby included herein by reference.

A sustained release of a second active agent may also be obtained by the use of a muco-adhesive polymeric device that is used intra-orally. When the drug is dispersed in a high viscosity polymeric device that will stick to, for example, the buccal membrane, release of the drug can be tuned to occur over several hours.

The invention further provides a kit of parts comprising a composition for immediate release of a first active agent and a composition for time-delayed immediate or sustained release of a second active agent. The kit preferably comprises instructions to administer said compositions at essentially the same time or said kit comprises instructions to first administrate the composition comprising a first active agent, followed by the administration of the composition for time-delayed immediate or sustained release of a second active agent.

In order to further enhance the effects of the kit of parts according to the invention, said kit may further comprise means for cognitive interventions and stimulation. Such information may be present on any data carrier (paper, CD, DVD), passive or interactive, or it may be a link to a website at least partially designed for the purpose of said cognitive stimulation. Sometimes it is preferred to present said cognitive stimulatory information subconsciously e.g. subliminally.

A time-delayed release composition, delays release of the drug and thereby also prevents release in the acidic environment of the stomach and allows release later in the gastrointestinal tract, for example in the more favorable environment of the small intestine or the colon. In addition to a time-delayed release composition, various materials, e. g., cellulose acetate phthalate, hydroxypropyl methylcellulose phthalate, polyvinyl acetate phthalate, and acrylic polymers, have been used as gastroresistant, enterosoluble coatings for delayed drug release in the intestine (Xu and Lee, Pharm. Res. 10 (8), 1144-1152 (1993)). Enteric coating systems, which are soluble at higher pH values, are frequently used for late intestinal and colon-specific delivery systems.

Said first active agent and second active agent are preferably selected from COMT inhibitors and/or male steroids. The first active agent and second active agent preferably are or comprise at least one male steroid such as testolactone, methyltesterone, fluoxymesterone, clostebol, formestan, methandriol dipropionate, methandrostenolone, methenolone, oxabolone, oxymesterone, testosterone and/or dihydrotestosterone. Said first and second active agent preferably are or comprise identical compounds that stimulate the dopamine and/or the noradrenergic system or, more preferably, are or comprise different compounds that stimulate the dopamine and/or the noradrenergic system. For example, said first agent preferably is or comprises testosterone and/or dihydrotestosterone and the second agent preferably is or comprises testolactone, methyltesterone, fluoxymesterone, clostebol, formestan, methandriol dipropionate, methandrostenolone, methenolone, oxabolone and/or oxymesterone.

Testosterone and/or dihydrotestosterone as a first agent is preferably administered in the form of a sublingual, buco-mucosal, or intranasal formulation comprising cyclodextrins as carrier, preferably hydroxypropyl-beta cyclodextrin. Said high anabolic compound is preferably present in a time-delayed release composition. It is preferred that the first and second agent are present in one pharmaceutical dosage form, preferably a tablet. Said first agent is preferably present in an outer layer that surrounds a core comprising the second agent..

Said composition comprising a first active agent and/or the composition comprising the second active agent preferably further comprises the selective estrogen-receptor modulator (SERM) that induces an adverse drug reaction, preferably tamoxifen. Tamoxifen is preferably provided with the inactive ingredients anhydrous lactose, colloidal silicon dioxide, crospovidone, magnesium stearate, microcrystalline cellulose and sodium lauryl sulfate.

### EXAMPLES

### Example 1

### Methods

2 healthy females performed a number of neuropsychological tests at baseline, after taking 40 mg/day of tamoxifen for 4 consecutive days, and after taking 40 mg/day of tamoxifen combined with 0.5 mg/day of sublingual testosterone for 4 consecutive days, to investigate any beneficial effects of sublingual testosterone in women taking a selective estrogen-receptor modulator (SERM).

The neuropsychological tests are:

Trail Making Test Parts A & B

Both parts of the Trail Making Test consist of 25 circles distributed over a sheet of paper. In Part A, the circles are numbered 1-25, and the patient should draw lines to connect the numbers in ascending order. In Part B, the circles include both numbers (1-13) and letters (A-L); as in Part A, the patient draws lines to connect the circles in an ascending pattern, but with the added task of alternating between the numbers and letters (i.e., 1-A-2-B-3-C, etc.). The patient should be instructed to connect the circles as quickly as possible, without lifting the pen or pencil from the paper.

15 Words Test

The 15 Words Test (15WT) is developed to investigate episodic memory problems in patients with brain disorders. It is the Dutch version of the Auditory Verbal Learning Test (REF?). This test consists of 15 words, which have to be learned during five trials. After every trial the respondent is asked to recall as many words as possible. After a distraction period of 20 minutes, the respondent is, again asked to name the words they have learned before.

Big/Little Circle (BLC)

### Task

Participants must first touch the smaller of the two circles displayed, then, after 20 trials, touch the larger circle for 20 further trials.

### Outcome Measures

This test has five outcome measures, covering latency (speed of response) and the participant's ability to touch the correct circle.

Intra-Extra Dimensional Set Shift (IED)

### Task

Two artificial dimensions are used in the test:
color-filled shapes
white lines

Simple stimuli are made up of just one of these dimensions, whereas compound stimuli are made up of both, namely white lines overlying colour-filled shapes. The participant starts by seeing two simple colour-filled shapes, and must learn which one is correct by touching it.

Feedback teaches the participant which stimulus is correct, and after six correct responses, the stimuli and/or rules are changed. These shifts are initially intra-dimensional (e.g. colour filled shapes remain the only relevant dimension), then later extra-dimensional (white lines become the only relevant dimension). Participants progress through the test by satisfying a set criterion of learning at each stage (six consecutive correct responses). If at any stage the participant fails to reach this criterion after 50 trials, the test terminates.

### Outcome Measures

This test has 18 outcome measures, assessing errors, and numbers of trials and stages completed.

Choice Reaction Time (CRT)

### Task

An arrow-shaped stimulus is displayed on either the left or the right side of the screen. The participant must press the left hand button on the press pad if the stimulus is displayed on the left hand side of the screen, and the right hand button on the press pad if the stimulus is displayed on the right hand side of the screen.

### Outcome Measures

This test has 13 outcome measures, assessing correct and incorrect responses, errors of commission and omission (late and early responses), and latency (response speed).

Match to Sample Visual Search (MTS)

### Task

The participant is shown a complex visual pattern (the sample) in the middle of the screen, and then, after a brief delay, a varying number of similar patterns is shown in a circle of boxes around the edge of the screen. Only one of these boxes matches the pattern in the center of the screen, and the participant must indicate which it is by touching it. Reaction time is measured on the basis of the release of the press-pad, which allows for its more accurate measurement.

### Outcome Measures

The outcome measures for this task cover numbers and percentages correct, and movement and reaction times

Reaction Time (RTI)

### Task

The task is divided into five stages, which require increasingly complex chains of responses. In each case, the participant must react as soon as a yellow dot appears. In some stages the dot may appear in one of five locations, and the participant must sometimes respond by using the press-pad, sometimes by touching the screen, and sometimes both.

### Outcome Measures

The four outcome measures in RTI are divided into reaction time (simple and 5-choice) and movement time (simple and five-choice).

Rapid Visual Information Processing (RVP)

### Task

A white box appears in the centre of the computer screen, inside which digits, from 2 to 9, appear in a pseudo-random order, at the rate of 100 digits per minute. Participants are requested to detect target sequences of digits (for example, 2-4-6, 3-5-7, 4-6-8) and to register responses using the press pad.

### Outcome Measures

The nine RVP outcome measures cover latency, probabilities and sensitivity.

### Results

In this proof of concept study, 2 healthy females performed a number of neuropsychological tests to investigate any beneficial effects of sublingual testosterone in women taking a SERM. The following neuropsychological domains were investigated (tasks are in brackets, see below for more information):
Information processing speed (Big/Little Circle, Choice Reaction Time, Match to Sample Visual Search, Trail making task A, 5-Choice Reaction Time)
Executive functioning (Intra-Extra Dimensional Set Shift, Trail making task B)
Reaction speed (Single Choice Reaction Time)
Working memory (Rapid Visual Information Processing)
Verbal memory (15-words task)

If tamoxifen in these women were to have detrimental effects on their cognitive functioning, a decrease in speed and/or accuracy on either of these tasks was expected. Tamoxifen, combined with sublingual testosterone, was tested to score an improved performance as compared to tamoxifen alone.

TABLE 1A

| | | | TAMOXIFEN + | |
|---|---|---|---|---|
| | BASELINE | TAMOXIFEN | T | |
| Subject ID | 1 | 1 | 1 | |
| Age | 45 | 45 | 45 | |
| BLC Percent correct | 100 | 100 | 100 | |
| BLC Mean correct latency | 450.75 | 390.9 | 386.725 | |
| CRT Mean correct latency | 256.7244898 | 243.54 | 225.3979592 | |
| CRT Percent incorrect trials | 2 | 0 | 2 | |
| MTS Mean correct latency | 1312.176471 | 1524.555556 | 1125.411765 | * |
| MTS Percent correct | 94.44444444 | 100 | 94.44444444 | |
| RTI Simple accuracy score | 15 | 14 | 15 | |
| RTI Simple reaction time | 303.1333333 | 308.3571429 | 258.6 | ** |
| RTI Five-choice accuracy score | 15 | 15 | 14 | |
| RTI Five-choice reaction time | 334.0666667 | 370.2666667 | 324.0714286 | * |
| Trail making A | 20.92 | 15.01 | 15.64 | |
| Trail making B | 28.28 | 23.72 | 23.86 | |
| 15 words direct | 42 | 51 | 58 | |
| 15 words recall | 7 | 6 | 9 | ** |
| 15 words recognition | 29 | 27 | 27 | |

TABLE 1B

| | | | TAMOXIFEN + | |
|---|---|---|---|---|
| | BASELINE | TAMOXIFEN | T | |
| Subject ID | 2 | 2 | 2 | |
| Age | 54 | 54 | 54 | |
| BLC Percent correct | 100 | 100 | 95 | |
| BLC Mean correct latency | 527.95 | 530.125 | 487.7105263 | * |
| CRT Mean correct latency | 288.84 | 280.84 | 265.54 | ** |
| CRT Percent incorrect trials | 0 | 0 | 0 | |
| MTS Mean correct latency | 1627.888889 | 1382.117647 | 1482.0625 | |
| MTS Percent correct | 100 | 94.44444444 | 88.88888889 | |
| RTI Simple accuracy score | 15 | 15 | 15 | |
| RTI Simple reaction time | 279.6666667 | 266.4666667 | 286.9333333 | |
| RTI Five-choice accuracy score | 15 | 15 | 15 | |
| RTI Five-choice reaction time | 277 | 287.6 | 268.0666667 | * |
| Trail making A | 18.19 | 18.36 | 18.37 | |
| Trail making B | 22.4 | 1.05.29 | 23.13 | *** |
| 15 words direct | 35 | 43 | 38 | |
| 15 words recall | 6 | 9 | 6 | |
| 15 words recognition | 28 | 28 | 27 | |

Tables 1A and 1B: Results from all tasks (except IED and RVP, which showed no effects) performed on baseline, and after 4 daily doses of 40 mg tamoxifen or 40 mg tamoxifen+0.5 mg testosterone. Latencies are in milliseconds, trail making test results are in seconds. 15 words test results shows number of recalled words (direct recall total number in 5 consecutive trials).

* denotes decreased performance in tamoxifen condition and increased performance in tamoxifen+t condition;
** denotes increased performance in tamoxifen+t condition;
***=if decreased performance in tamoxifen condition, then normalized performance in tamoxifen+t condition (tamoxifen result is unreliable due to insufficient task instruction. Please note that the reaction time in the tamoxifen condition is 1 minute, 5.29 seconds).

The results clearly show multiple indications of deteriorating effects of tamoxifen and positive effects of added testosterone in both subjects regarding information processing speed.

An indication of deteriorating effects of tamoxifen and positive effects of added testosterone in 1 subject (trail making B) regarding executive functioning.

A positive effect (15 words recall) of added testosterone was seen in 1 subject regarding verbal memory.

No effects were found regarding reaction speed and working memory.

1. A method of treatment of an individual suffering from or at risk of suffering from a selective estrogen-receptor modulator (SERM) induced adverse drug reaction, said method comprising administering to the individual in need thereof a compound that stimulates the dopamine and/or the noradrenergic system in the individual.

2. The method of claim 1, wherein the adverse drug reaction includes depression, decreased sexual desire, cognitive side effects and/or arthralgic symptoms.

3. The method of claim 1, wherein the compound is a catechol-O-methyltransferase (COMT) inhibitor.

4. The method of claim 1, wherein the compound is an estrogen.

5. The method of claim 1, wherein the compound is a male steroid.

6. The method of claim 5, wherein the steroid is administered such that the circadian rhythm of testosterone is mimicked.

7. The method of claim 5, wherein the administering comprises:
administering said male steroid daily at a specific time of the day in a composition that is designed for immediate peak release of said steroid into the bloodstream, and
administering said male steroid in a composition for time-delayed immediate or sustained release of said steroid,
wherein the first and second mail steroids comprise a high androgenic and/or a high anabolic steroid.

8. The method of claim 1, wherein the SERM is administered to the individual in the course of a breast cancer treatment.

9. The method of claim 8, wherein the individual is suffering from or at risk of suffering from breast cancer metastasis.

10. The method of claim 1, further comprising the diagnosis of depression in said individual prior to and/or during said treatment.

11. The method of claim 10, wherein said diagnosis comprises decreased mood and/or decreased sexual desire and/or cognitive side effects and/or muscle ache.

12. The method of claim 1, wherein the SERM is an estrogen antagonist in breast tissue.

13. The method of claim 1, wherein the individual is further receiving an aromatase inhibitor.

14. (canceled)

15. A pharmaceutical dosage form comprising a composition for pulsatile, not-continuous release of a compound or a combination of compounds that stimulate the dopamine and/or the noradrenergic system.

16. A tablet comprising a composition for immediate sublingual release of a first active agent and a composition for delayed immediate or sustained release of a second active agent, wherein the first active agent and the second active agent is a COMT inhibitor or a male steroid.

17. A tablet according to claim 16, wherein the composition for delayed immediate or sustained release comprises testolactone, methyltesterone, fluoxymesterone, clostebol, formestan, methandriol dipropionate, methandrostenolone, methenolone, oxabolone and/or oxymesterone.

18. A kit of parts comprising a composition for immediate release of a first active agent and a composition for delayed immediate or sustained release of a second active agent, wherein the first active agent and/or the second active agent is a male steroid.

19. The method of claim 4, wherein the estrogen is estradiol or androstanediol.

20. The method of claim 5, wherein the male steroid is testosterone and/or dihydrotestosterone.

21. The method of claim 7, wherein the male steroid is testosterone and/or dihydrotestosterone.

22. The method of claim 12, wherein the estrogen antagonist is tamoxifen.

23. A tablet according to claim 16, wherein the male steroid is testosterone or dihydrotestosterone.

24. The kit of parts according to claim 18, wherein the male steroid is testosterone or dihydrotestosterone.
